(19) United States
(12) Patent Application Publication
(10) Pub. No.: US 2009/0054931 A1
(43) Pub. Date: Feb. 26, 2009

(54) Spline Implant

(76) Inventor: Peter Metz-Stavenhagen, Bad Wildungen (DE)

Correspondence Address: PYLE & PIONTEK;ATTN: THOMAS R. VIGIL, 221 N LASALLE STREET , ROOM 2036, ROOM 2036, CHICAGO, IL, 60601, US

(21) Appl. No.: 12/224,732

(22) PCT Filed: Apr. 18, 2007
(86) PCT No.: PCT/DE2007/000670

§ 371 Date: Sep. 4, 2008

(30) Foreign Application Priority Data
Apr. 29, 2006 (DE) 20 2006 006 898.8

(51) Int. Cl. A61B 17/70; A61F 2/44

(52) U.S. Cl. 606/248; 623/17.11

(57) ABSTRACT

The invention relates to a vertebral implant that is to be mounted between the lamina and/or the spinous processes of adjoining vertebral bodies (12, 12′). In order to create a vertebral implant of said type, a bottom part (11) is provided that is to be mounted on a first vertebral body (12′) while a top part (10) is provided which is to be mounted on a second vertebral body (12). A contact surface (13, 14), by means of which the bottom part (11) and the top part (10) rest against each other, is embodied on both the bottom part (11) and the top part (10).

The present invention relates to a spine implant for vertebral attachment between the spinal processes of neighboring vertebral bodies. The vertebrae of the spine not only include the cervical, dorsal and lumbar vertebrae but also the sacrum.

From CA 2,320,821 and EP 1 330 987 B1 there are known interspinous spine implants for vertebral placement between neighboring spinal processes of the neighboring vertebral bodies. These spine implants include a spring element that is interposed between the spinal processes and four holding webs laterally abutting the spinal processes in order to keep the spine implant in the respective position. It is not necessary to fasten this spine implant to the vertebral body since the spring element keeps the entire spine implant in position by virtue of its tensioning force. The four retaining webs merely serve to secure the spine implant from sliding laterally out of place.

In case of kyphosis, it repeatedly happens that parts of the vertebral bone are damaged with time due to the high prevailing pressures. It may also happen that nerves get trapped between the vertebral bones, causing great pain.

To avoid such kyphosis it is necessary to space neighboring vertebral bodies apart from each other, at least on the vertebral side. The spine implant known from CA 2,320,821 and EP 1 330 987 B1 is only conditionally suited since the respective spring element is compressed to a very large extent when subjected to high occurring forces so that the desired distraction gets lost. Also, the spring elements act in such a manner onto the spinal processes of the neighboring vertebral bones that their freedom of movement is impaired.

In view thereof, it is the object of the present invention to provide a spine implant of the type mentioned herein above by means of which a kyphosis can be reliably prevented and that allows for great freedom of movement of the vertebral bones.

As a technical solution to this object, there is proposed, in accordance with the features of claim 1, a spine implant. Advantageous further developments of this spine implant are recited in the dependent claims.

A spine implant configured according to this technical teaching has the advantage that the bottom part attached to a first vertebral body and the top part attached to a second vertebral body spaces the vertebral bodies a defined distance apart so that a defined spaced-apart relationship of the neighboring vertebral bodies is achieved. This is particularly advantageous in case of damaged bone portions, in particular on the joint processes, since these can be completely relieved by this measure. It is understood that the attending physician has at his disposal a plurality of bottom and top parts, each of a different thickness so that he can choose, depending on the findings, matching bottom and top parts for achieving the desired spacing between the vertebral bones.

In many cases it is advantageous not to attach the spine implant between the spinal processes like in prior art but on a lamina of the vertebral arch instead, in particular if the spinal processes are damaged in case of a kyphosis or the like. Such an interlaminar attachment of the spine implant to a lamina also offers the advantage that the lever forces there are not so high so that the vertebral bone is relieved. It is understood that even if the spine implant is disposed in the region of the lamina the vertebral bones are spaced apart durably and reliably.

Another advantage is that a hypomochlion is possible with the spine implant of the invention being placed in the lamina. Neighboring vertebral bones are distracted by the spine implant and concurrently compressed through a clamp attached vertically to the neighboring vertebral body so that the spine is lordosed.

In a particularly preferred embodiment, the contact surfaces of the spine implant are arranged substantially horizontally. The advantage thereof is that the forces acting onto the implant are introduced almost at right angles to the contact surface so that they can well be received. Another advantage is that the discrete vertebral bodies remain capable of lateral movement since horizontal arrangement of the contact surface does not hinder this intended movement.

In a preferred developed implementation, the two contact surfaces are configured to be planar so that a lateral movement of the vertebral bones is not opposed.

In another preferred embodiment, one contact surface is configured to be level and the other one convex. As a result, the vertebral bones continue to be capable of lateral movement and concurrently the spine implant allows for relative rotation of the vertebral bones so that the neighboring vertebral bones can be moved in all the directions.

In still another preferred embodiment, one contact surface is convex and the other one concave. As a result, the two contact surfaces abut on each other over a large surface, the vertebral bones and as a result thereof the spine remaining very flexible in this case as well. Another advantage is that sagittal displacements are avoided.

As an alternative thereto, one contact surface can be configured to be fluted instead of concave.

In a particularly preferred embodiment, webs are formed integral with the bottom and/or top part, said webs serving to fasten the spine implant to the vertebral bone. These webs are configured such that they laterally abut the spinal processes or the lamina. In a preferred developed implementation, each bottom and top part comprises two such webs, the spine implant having in this case a substantially U-shaped appearance in cross section. It has been found advantageous to configure one web shorter than the other one for ease of insertion of the spine implant between the vertebral bodies.

Further advantages of the spine implant of the invention will become apparent in the appended drawings and in the following description of embodiments thereof. Likewise, the invention lies in each and every novel feature or combination of features mentioned above or described herein after. The embodiments discussed herein are merely exemplary in nature and are not intended to limit the scope of the invention in any manner. In said drawing:

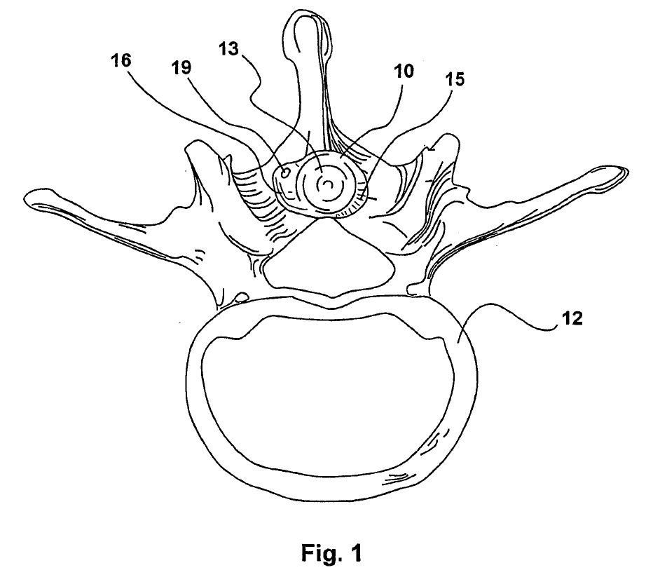

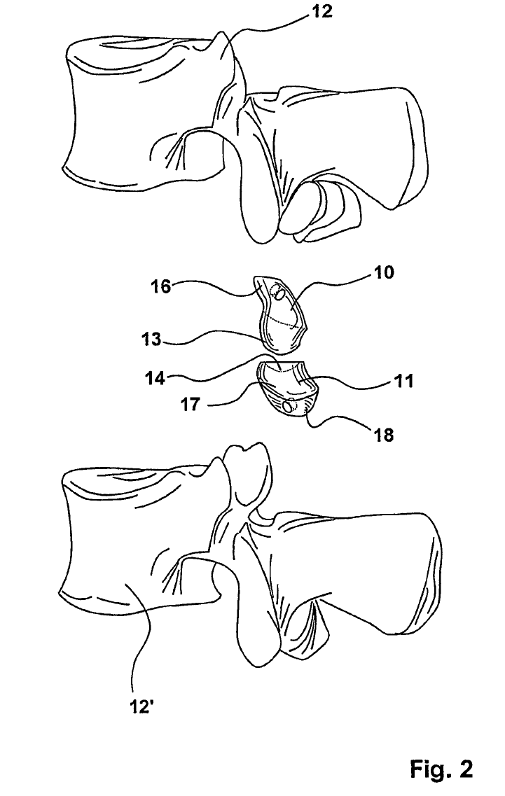

FIG. 1 is a top view of a vertebral body with a bottom part of a spine implant as shown in FIG. 2;

FIG. 2 is an exploded side view of a spine implant of the invention placed into the spine;

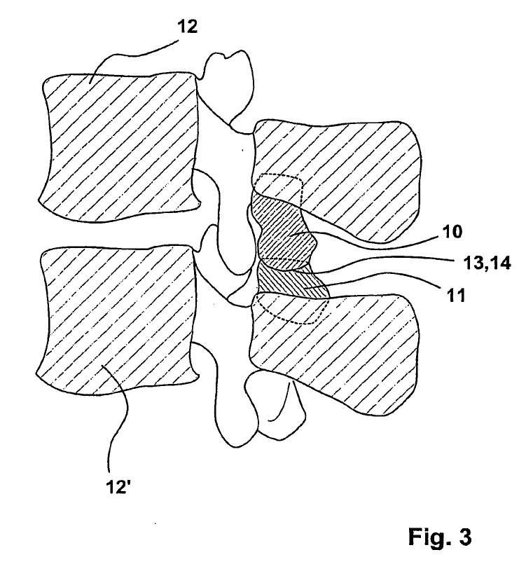

FIG. 3 is a sectional side view of the spine implant shown in FIG. 2, implanted between the laminae of neighboring vertebrae;

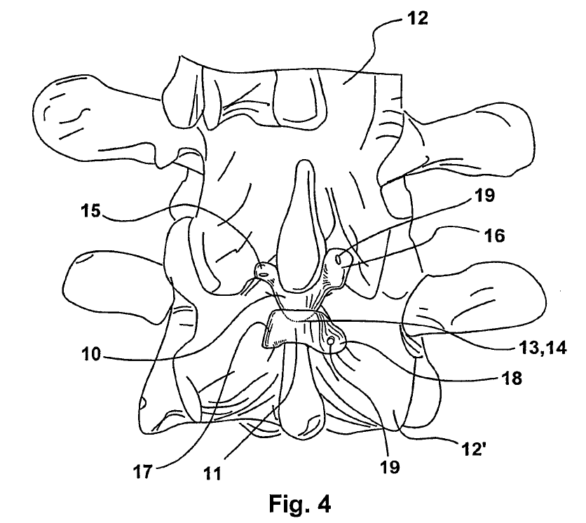

FIG. 4 is a back view of the spine implant shown in FIG. 2, implanted between the laminae of neighboring vertebrae;

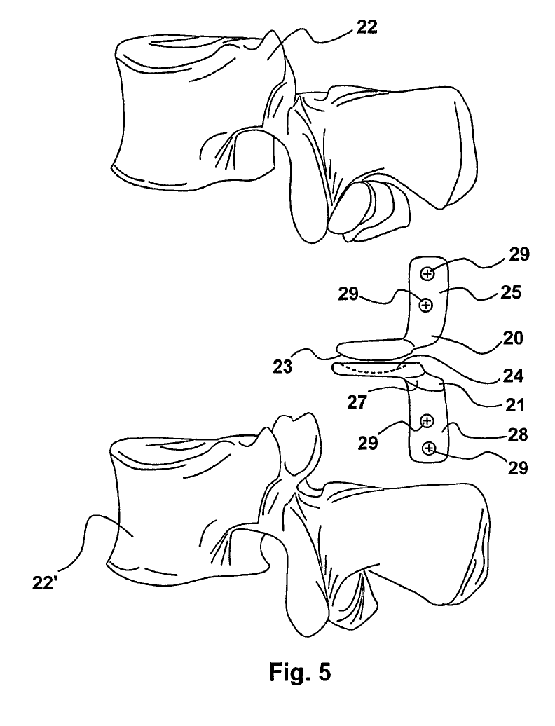

FIG. 5 is an exploded side view of a second embodiment of a spine implant of the invention inserted in the spine between the spinal processes;

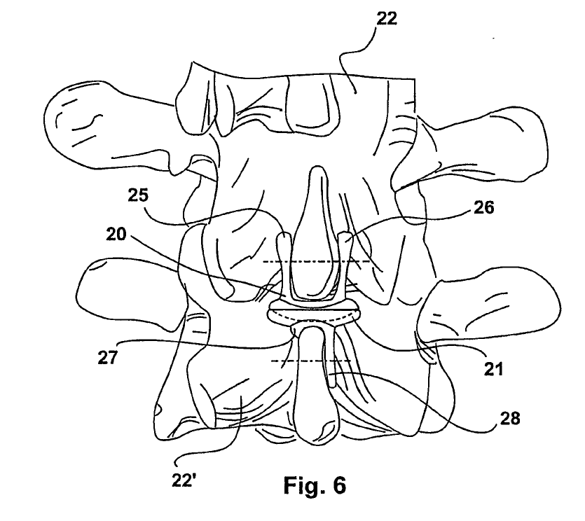

FIG. 6 is a back view of the spine implant shown in FIG. 5, implanted in the spine between the spinal processes;

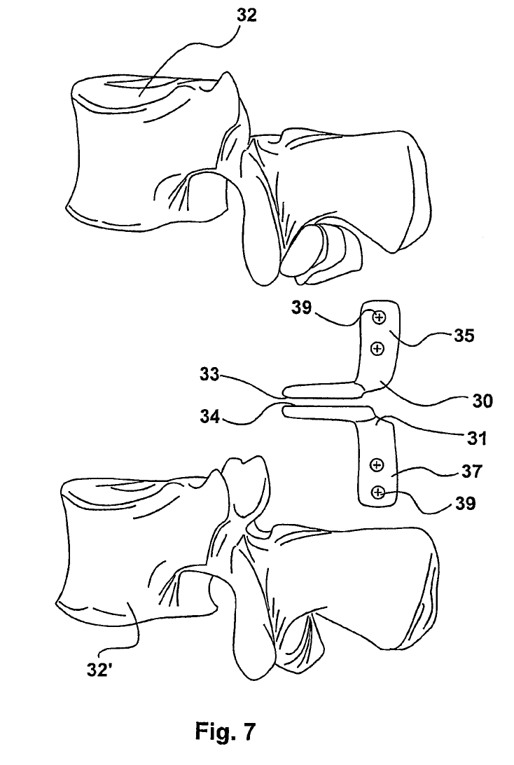

FIG. 7 is an exploded side view of a third embodiment of a spine implant of the invention inserted in the spine between the spinal processes;

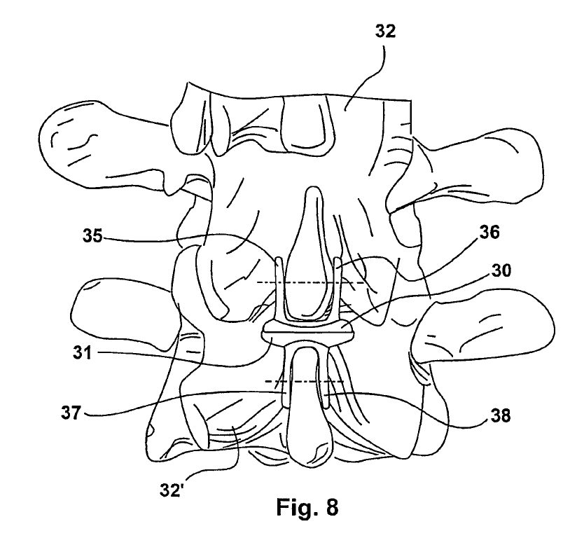

FIG. 8 is a back view of the spine implant shown in FIG. 7, implanted in the spine between the spinal processes;

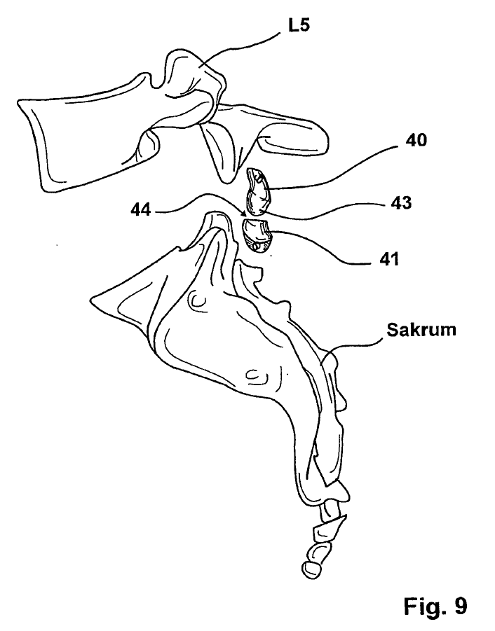

FIG. 9 is an exploded side view of a fourth embodiment of a spine implant of the invention inserted between the spinal processes of the fifth lumbar vertebra and the sacrum;

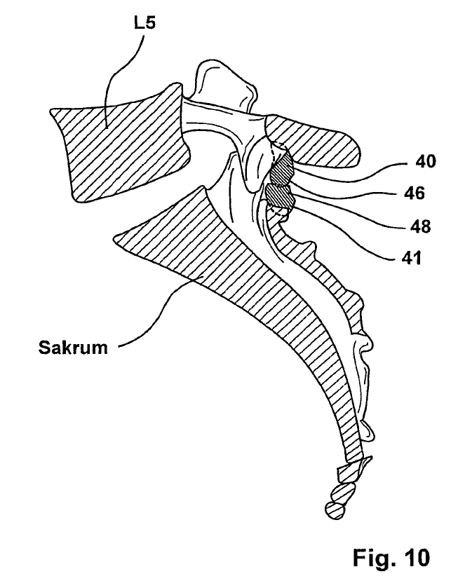

FIG. 10 is a sectional side view of the spine implant shown in FIG. 9, implanted between the lamina of L5 and the sacrum;

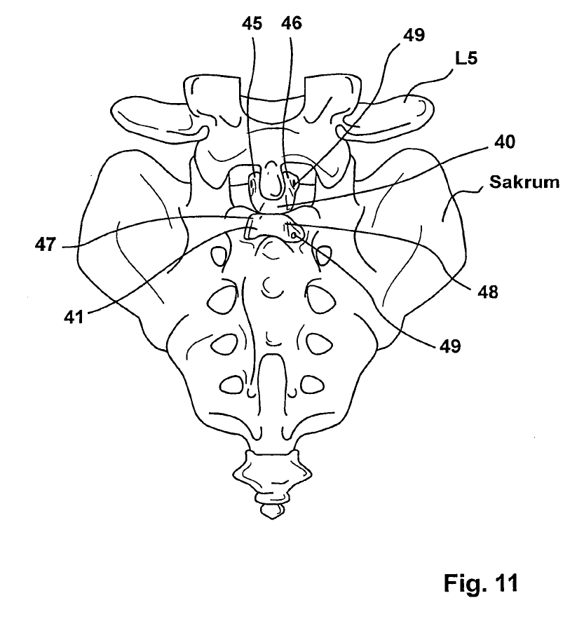

FIG. 11 is a back view of the spine implant shown in FIG. 9, implanted between the spinal processes of L5 and the sacrum.

In the FIGS. 1 through 4, there is illustrated a first embodiment of an interlaminar spine implant including a top part 10 and a bottom part 11. These parts are attached on the vertebral side between a first vertebral body 12 and a neighboring second vertebral body 12′ in the region of the respective vertebral arches.

The top part 10 has a convex shaped contact surface 13 and the bottom part 11 has a mating concave contact surface 14. The top part 10 and the bottom part 11 are attached to the respective vertebral body 12, 12′ for the contact surface 13 of the top part 10 to engage the contact surface 14 of the bottom part 11 over a large surface. It has been found advantageous to arrange the convex contact surface 13 and the concave contact surface 14 substantially horizontally in order for them to best be capable of taking the occurring forces.

On the top part 10, a short web 15 and a long web 16 project from the contact surface 13, said webs abutting the lamina of the vertebral arch of the vertebral body 12 in order to fix the top part 10. Analogous applies to the bottom part 11. Here also there are provided a projecting short web 17 and a projecting long web 18 which also abut the lamina of the vertebral arch of the vertebral body 12′ to fix the bottom part 11 there. In the long webs 16, 18 there is provided a cutout 19 for a nail or a screw for affixing the top part 10 and the bottom part 11 to the vertebral body 12, 12′ to engage.

Through the relative proximity of the lamina to the center of the vertebral body, the lever forces which occur here are not so high so that the load on the vertebral bone is less. Moreover, the vertebral body 12′ is much more stable in the region of the vertebral arch so that the vertebral body 12′ is prevented from being injured. Another advantage is that the available surface of the bone is much larger here so that the occurring forces can be distributed over a larger area, this in turn reducing the load.

Through the convex/concave configuration of the contact surfaces 13, 14, one obtains the greatest possible flexibility of the vertebral bodies 12, 12′ with respect to each other so that the natural flexibility of the locomotory system is not affected by the spine implant. By virtue of the friction between the two contact surfaces 13, 14 resorption of the bone through conventional rigid spacers (interspinous implants) is avoided. Another advantage is that, since the contact surfaces 13, 14 are displaceable with respect to each other, they are prevented from being displaced on a sagittal plane. It should not be forgotten that the vertebral bodies 12, 12′ are durably spaced apart in this part thanks to the top part 10 and to the bottom part 11, thus preventing the formation of a kyphosis and durably freeing possibly trapped nerves.

In the FIGS. 5 and 6, there is shown a second embodiment of a spine implant of the invention, which also includes a top part 20 and a bottom part 21. As contrasted to the first interlaminar spine implant, this second embodiment is placed between the spinal processes. Here also, the contact surfaces 23 and 24 are configured to be concave and convex respectively. Unlike the first embodiment shown in the FIGS. 1 through 4, the webs 25, 26, 27, 28 of the top part 20 and of the bottom part 21 are configured to be much longer, each comprising two cutouts 29 for receiving a nail or a screw. As a result, the spine implant is suited for attachment to the spinal processes of neighboring vertebral bodies 22, 22′ whilst the spine implant shown in the FIGS. 1 through 3 is devised for attachment to the laminae of the respective vertebral bodies 12, 12′.

The third embodiment shown in the FIGS. 7 and 8 merely differs from the second embodiment shown in the FIGS. 5 and 6 by the fact that here the two contact surfaces 33 and 34 of the top part 30 and of the bottom part 31 are each configured to be level.

In the FIGS. 9 through 11, there is shown a fourth embodiment of a spine implant of the invention which is interposed between the fifth lumbar vertebra and the sacrum. This spine implant rests on the lamina of the two vertebral bodies. This spine implant according to the fourth embodiment also includes a top part 40 that is fastened to the lumbar vertebra L5 and a bottom part 41 attached to the sacrum. The top part 40 also has a convex contact surface 43 whilst the bottom part 41 has a concave contact surface 44. The top part 40 has two projecting webs 45, 46 and the bottom part 41 has two projecting webs 47, 48, web 48 being configured to be slightly longer than web 47. By means of these webs 45, 46, 47, 48 both the top part 40 and the bottom part 41 can be fastened to the vertebral body L5 or to the sacrum by means of nails or screws which can be threaded through a corresponding cutout 49.

In another embodiment that has not been illustrated herein, one contact surface, instead of being concave, is configured to be fluted, the other, convex contact surface abutting the fluted contact surface.

### List of Numerals:

| | | | | |
|---|---|---|---|---|
| 10 | 20 | 30 | 40 | top part |
| 11 | 21 | 31 | 41 | bottom part |
| 12 | 22 | 32 | L5 | vertebral body |
| 12′ | 22′ | 32′ | sacrum | vertebral body |
| 13 | 23 | 33 | 43 | contact surface |
| 14 | 24 | 34 | 44 | contact surface |
| 15 | 25 | 35 | 45 | web |
| 16 | 26 | 36 | 46 | web |
| 17 | 27 | 37 | 47 | web |
| 18 | 28 | 38 | 48 | web |
| 19 | 29 | 39 | 49 | cutout |

1. A spine implant for vertebral attachment between the laminae and/or the spinal processes of neighboring vertebral bodies (12, 12′, 22, 22′, 32, 32′, L5, sacrum),
characterized by
a bottom part (11, 21, 31, 41) for attachment to a first vertebral body (12′, 22′, 32′, L5, sacrum) and by a top part (10, 20, 30, 40) for attachment to a second vertebral body (12, 22, 32, L5, sacrum), one contact surface (13, 14, 23, 24, 33, 34, 43, 44) at which said bottom part (11, 21, 31, 41) and said top part (10, 20, 30, 40) abut each other being configured respectively both on said bottom part (11, 21, 31, 41) and on said top part (10, 20, 30, 40).

2. The spine implant as set forth in claim 1,
characterized in
that the contact surfaces (13, 14, 23, 24, 33, 34, 43, 44) are arranged substantially horizontally.

3. The spine implant as set forth in claim 1,
characterized in
that at least one contact surface (33, 34) is
configured to be level.

4. The spine implant as set forth in claim 1,
characterized in
that at least one contact surface (13, 23, 43) is configured to be convex.

5. The spine implant as set forth in claim 4,
characterized in
that one contact surface (13, 23, 43) is configured to be convex and that the other contact surface (14, 24, 44) is configured to be correspondingly concave.

6. The spine implant as set forth in claim 4,
characterized in
that one contact surface is configured to be convex and that the other contact surface is configured to be correspondingly fluted.

7. The spine implant as set forth in claim 1,
characterized in
that at least one web (15, 16, 17, 18, 25, 26, 27, 28, 35, 36, 37, 38, 45, 46, 47, 48) is formed respectively on the bottom part (11, 21, 31, 41) and/or on the top part (10, 20, 30, 40), said web abutting laterally the spinal process or the lamina.

8. The spine implant as set forth in claim 7,
characterized in
that a cutout (19, 29, 39, 49) for receiving a
screw or a nail is provided in the web (15, 16, 17, 18, 25, 26, 27, 28, 35, 36, 37, 38, 45, 46, 47, 48).

9. The spine implant as set forth in claim 7,
characterized in
that two webs (25, 26, 27, 28) are arranged on the bottom part (21) and/or on the top part (20) so as to face each other in such a way that said webs (25, 26, 27, 28) form, together with the respective contact surface (13, 14, 23, 24), a surrounding, preferably U-shaped, grip about the spinal process or the lamina.

10. The spine implant as set forth in claim 9,
characterized in
that one web (15,17, 45, 47) is shorter than the other web (16, 18, 46, 48).

11. The spine implant as set forth in claim 8,
characterized in
that two webs (25, 26, 27, 28) are arranged on the bottom part (21) and/or on the top part (20) so as to face each other in such a way that said webs (25, 26, 27, 28) form, together with the respective contact surface (13, 14, 23, 24), a surrounding, preferably U-shaped, grip about the spinal process or the lamina.
